(19) United States
(12) Patent Application Publication
(10) Pub. No.: US 2026/0069365 A1
(43) Pub. Date: Mar. 12, 2026

(54) DYNAMIC SELECTION AND MANAGEMENT OF DISPLAYED PREOPERATIVE AND INTRAOPERATIVE IMAGE DATA OF A SCENE

(71) Applicant: Proprio, Inc., Seattle, WA (US)

(72) Inventors: Nicholas Matthew Miclette, Seattle, WA (US); Camille Cheli Farley, Kirkland, WA (US); David Lee Fiorella, Seattle, WA (US); Kenan Chabuk, Seattle, WA (US); Andrew Dean, Seattle, WA (US)

(21) Appl. No.: 19/321,043

(22) Filed: Sep. 5, 2025

Related U.S. Application Data
(60) Provisional application No. 63/692,032, filed on Sep. 7, 2024.

(51) Int. Cl. A61B 34/20; A61B 90/00; G06T 7/00; G06T 7/11; G06T 19/00

(52) U.S. Cl. CPC A61B 34/20; G06T 7/0012; G06T 7/11; G06T 19/006; A61B 2034/2065; A61B 2090/3762; G06T 2200/24; G06T 2207/10081; G06T 2207/30012; G06T 2210/12; G06T 2210/41

(57) ABSTRACT

Systems, methods, and devices for selecting and managing imaging systems, such as surgical imaging systems, are disclosed herein. A representative method can include detecting the introduction of a first instrument into a bounding region around a vertebra and designating the first instrument as an active instrument. The active instrument can be used to control the display of imaging data on a user interface. The method can further include detecting that the first instrument has left the bounding region while a second instrument has entered the bounding region, and designating the second instrument as the active instrument. Another representative method can include defining multiple bounding regions around corresponding vertebra, detecting the entry of the active instrument into one of the bounding regions, and designating the vertebra associated with the bounding region as an active vertebra for further control of the display of imaging data on the user interface.

### CROSS-REFERENCE TO RELATED APPLICATION(S)

The present application claims the benefit of U.S. Provisional Patent Application No. 63/692,032, titled “DYNAMIC SELECTION AND MANAGEMENT OF DISPLAYED PREOPERATIVE AND INTRAOPERATIVE IMAGE DATA OF A SCENE,” and filed Sep. 7, 2024, which is incorporated herein by reference in its entirety

### TECHNICAL FIELD

The present technology generally relates to methods and systems for dynamically managing the display of previously-captured image data, such as preoperative medical images (e.g., computed tomography (CT) scan data), during a surgical procedure.

### BACKGROUND

In a mediated reality system, an image processing system adds, subtracts, and/or modifies visual information representing an environment. For surgical applications, a mediated reality system may enable a surgeon to view a surgical site from a desired perspective together with contextual information that assists the surgeon in more efficiently and precisely performing surgical tasks. When performing surgeries, surgeons often rely on preoperative three-dimensional (3D) images of the patient's anatomy, such as CT scan images. However, the usefulness of such preoperative images is limited because the images cannot be easily integrated into the operative procedure. For example, to make use of the preoperative images during the surgery, the surgeon must manually designate the particular instrument and/or vertebra to view the corresponding preoperative images during the procedure, which can lead to errors in identifying the correct instrument and/or vertebra. Additionally, the need for continuous manual adjustments may also require significant attention that takes away from the surgeon's focus on the operation and thus may disrupt the workflow and prolong the procedure.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1 is a schematic view of an imaging system in accordance with embodiments of the present technology.

FIG. 2 is a perspective view of an environment employing the imaging system of FIG. 1 in accordance with embodiments of the present technology.

FIG. 3 is an isometric view of a portion of the imaging system of FIG. 1 illustrating four cameras of a sensor array of the imaging system in accordance with embodiments of the present technology.

FIGS. 4A and 4B are an isometric view and a cross-sectional view, respectively, of a tracking environment imaged/sensed by the system of FIG. 1 in accordance with embodiments of the present technology.

FIG. 5 is a flow diagram of a process or method for dynamically selecting and managing an active instrument in the tracking environment of FIGS. 4A and 4B in accordance with embodiments of the present technology.

FIGS. 6A-6C illustrate a user interface visible to a user of the system of FIG. 1 for displaying imaging data according to the method of FIG. 5 in accordance with embodiments of the present technology.

FIG. 7 is an isometric view of a tracking environment imaged/sensed by the system of FIG. 1 in accordance with embodiments of the present technology.

FIG. 8 is a flow diagram of a process or method for dynamically selecting and managing an active vertebra after detecting an active instrument in the tracking environment of FIG. 7 in accordance with embodiments of the present technology.

FIGS. 9A and 9B illustrate a user interface visible to a user of the system of FIG. 1 for displaying imaging data according to the method of FIG. 8 in accordance with embodiments of the present technology.

### DETAILED DESCRIPTION

Aspects of the present technology are directed generally to a system for managing image guided-navigation systems (e.g., augmented-reality imaging systems, virtual-reality imaging systems, mediated-reality imaging systems), such as for use in surgical procedures, and associated devices methods. In some embodiments, the imaging system includes (i) a camera array including a plurality of cameras configured to capture intraoperative image data (e.g., light field data, RGB, and/or depth data) of a surgical scene and (ii) a processing device communicatively coupled to the camera array. The camera array can further include one or more trackers configured to track one or more tools (e.g., instruments) through the surgical scene. The processing device can be configured to synthesize/generate a three-dimensional (3D) virtual image corresponding to a virtual perspective of the scene in real-time or near-real-time based on the image data from at least a subset of the cameras. The processing device can output the 3D virtual image to a display device (e.g., a head-mounted display (HMD)) for viewing by a viewer, such as a surgeon or other operator of the imaging system. The imaging system is further configured to receive and/or store preoperative image data. The preoperative image data can be medical scan data (e.g., computerized tomography (CT) scan data) corresponding to a portion of a patient in the scene, such as a spine of a patient undergoing a spinal surgical procedure. The processing device can register the preoperative image data to the intraoperative image data by, for example, registering/matching fiducial markers and/or other feature points visible in 3D data sets representing both the preoperative and intraoperative image data. The processing device can further display the preoperative image on the display device along with a representation of the tool. This can allow a user, such as a surgeon, to simultaneously view the underlying 3D anatomy of a patient undergoing an operation and the position of the tool relative to the 3D anatomy.

In several of the embodiments described below, the system can define a bounding region around a vertebra and detect the introduction of a first instrument into the bounding region. Upon detecting the first instrument, the system can designate the first instrument as the active instrument. The active instrument can control display of preoperative, intraoperative, and/or other imaging data on a display of the system. When a second instrument is detected entering the same bounding region while the first instrument is still within the bounding region, the system can maintain the first instrument as the active instrument. If the first instrument is detected outside of the bounding region while the second instrument is within the bounding region, the system can designate the second instrument as the active instrument for control of the imaging data on the display.

In several of the embodiments described below, the system can define bounding regions around corresponding vertebra, detect the entry of the active instrument into one of the bounding regions, and designate the vertebra associated with the bounding region as an active vertebra for further control of the display of imaging data on the user interface. More specifically, the system can detect the active instrument entering a first bounding region around a first vertebra and can designate the first vertebra as the active vertebra in response to the entry of the instrument. When the active instrument enters a second bounding region around a second vertebra, which is located outside the first bounding region, the system can designate the second vertebra as the active vertebra in response to detecting the instrument in the second bounding region. The system can display 3D image data including a representation of the identified active instrument and active vertebra on the display of the system.

The system provides certain benefits in several of the embodiments described below, particularly in improving the precision and efficiency of surgical procedures. By dynamically selecting and managing 3D and/or 2D virtual perspectives of a surgical scene in real-time or near-real-time, the system eliminates the need for manual adjustments of the imaging system, thereby reducing the risk of errors and allowing the viewer of the imaging system to maintain focus on the operation. The ability of the system to track surgical instruments and vertebrae automatically provides the most relevant views of the surgical site, which can further improve the efficiency of the procedure.

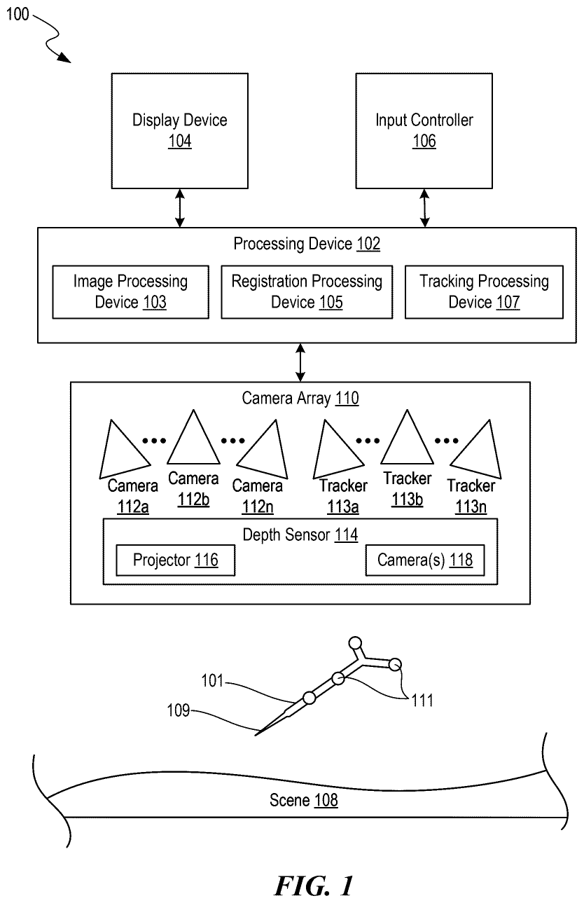

Specific details of several embodiments of the present technology are described herein with reference to FIGS. 1-11. The present technology, however, can be practiced without some of these specific details. In some instances, well-known structures and techniques often associated with image displays, optical tracking, user interfaces, and the like have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section.

Moreover, although frequently described in the context of displaying preoperative image data and/or intraoperative image data of a spinal surgical scene, the methods and systems of the present technology can be used to display image data of other types. For example, the systems and methods of the present technology can be used more generally to display any previously-captured image data of a scene to generate a mediated reality view of the scene including a fusion of the previously-captured data and real-time images.

The accompanying figures depict embodiments of the present technology and are not intended to be limiting of its scope. Depicted elements are not necessarily drawn to scale, and various elements can be arbitrarily enlarged to improve legibility. Component details can be abstracted in the figures to exclude details as such details are unnecessary for a complete understanding of how to make and use the present technology. Many of the details, dimensions, angles, and other features shown in the Figures are merely illustrative of particular embodiments of the disclosure. Accordingly, other embodiments can have other dimensions, angles, and features without departing from the spirit or scope of the present technology.

The headings provided herein are for convenience only and should not be construed as limiting the subject matter disclosed. To the extent any materials incorporated herein by reference conflict with the present disclosure, the present disclosure controls.

### I. Selected Embodiments of Imaging Systems

FIG. 1 is a schematic view of an imaging system 100 (“system 100”) in accordance with embodiments of the present technology. In some embodiments, the system 100 can be a synthetic augmented reality system, a virtual-reality imaging system, an augmented-reality imaging system, a mediated-reality imaging system, and/or a non-immersive computational imaging system. In the illustrated embodiment, the system 100 includes a processing device 102 that is communicatively coupled to one or more display devices 104, one or more input controllers 106, and a sensor array 110 (e.g., a camera array, a sensor head, and/or the like). In other embodiments, the system 100 can comprise additional, fewer, or different components. In some embodiments, the system 100 includes some features that are generally similar or identical to those of the mediated-reality imaging systems disclosed in (i) U.S. patent application Ser. No. 16/586,375, filed Sep. 27, 2019, titled “CAMERA ARRAY FOR A MEDIATED-REALITY SYSTEM,” and/or (ii) U.S. patent application Ser. No. 15/930,305, filed May 12, 2020, and titled “METHODS AND SYSTEMS FOR IMAGING A SCENE, SUCH AS A MEDICAL SCENE, AND TRACKING OBJECTS WITHIN THE SCENE,” each of which is incorporated herein by reference in its entirety.

In the illustrated embodiment, the sensor array 110 includes a plurality of cameras 112 (identified individually as cameras 112a-112n; which can also be referred to as first cameras) that can each capture images of a scene 108 (e.g., first image data) from a different perspective. The scene 108 can include for example, a patient undergoing surgery (e.g., spinal surgery) and/or another medical procedure. In other embodiments, the scene 108 can be another type of scene. The sensor array 110 can further include dedicated object tracking hardware (e.g., including individually identified trackers 113a-113n) that captures positional data of one more objects, such as an instrument 101 (e.g., a surgical instrument or tool) having a tip 109, to track the movement and/or orientation of the objects through/in the scene 108. In some embodiments, the cameras 112 and the trackers 113 are positioned at fixed locations and orientations (e.g., poses) relative to one another. For example, the cameras 112 and the trackers 113 can be structurally secured by/to a mounting structure (e.g., a common frame) at predefined fixed locations and orientations. In some embodiments, the cameras 112 are positioned such that neighboring cameras 112 share overlapping views of the scene 108. In general, the position of the cameras 112 can be selected to maximize clear and accurate capture of all or a selected portion of the scene 108. Likewise, the trackers 113 can be positioned such that neighboring trackers 113 share overlapping views of the scene 108. Therefore, all or a subset of the cameras 112 and the trackers 113 can have different extrinsic parameters, such as position and orientation (e.g., pose).

In some embodiments, the cameras 112 in the sensor array 110 are synchronized to capture images of the scene 108 simultaneously (within a threshold temporal error). In some embodiments, all or a subset of the cameras 112 are light field, plenoptic, and/or RGB cameras that capture information about the light field emanating from the scene 108 (e.g., information about the intensity of light rays in the scene 108 and also information about a direction the light rays are traveling through space). In some embodiments, image data from the cameras 112 can be used to reconstruct a light field of the scene 108. More specifically, the cameras 112 can be RGB cameras that capture a combined image data set for reconstructing a light field of the scene 108. Therefore, in some embodiments the images captured by the cameras 112 encode depth information representing a surface geometry of the scene 108. In some embodiments, the cameras 112 are substantially identical. In other embodiments, the cameras 112 include multiple cameras of different types. For example, different subsets of the cameras 112 can have different intrinsic parameters such as focal length, sensor type, optical components, and the like. The cameras 112 can have charge-coupled device (CCD) and/or complementary metal-oxide semiconductor (CMOS) image sensors and associated optics. Such optics can include a variety of configurations including lensed or bare individual image sensors in combination with larger macro lenses, micro-lens arrays, prisms, and/or negative lenses. For example, the cameras 112 can be separate light field cameras each having their own image sensors and optics. In other embodiments, some or all of the cameras 112 can comprise separate microlenslets (e.g., lenslets, lenses, microlenses) of a microlens array (MLA) that share a common image sensor. In other embodiments, some or all of the cameras 112 can be RGB (e.g., color) cameras having visible imaging sensors that together provide a light field data set of the scene 108.

In some embodiments, the trackers 113 are imaging devices, such as infrared (IR) cameras that can capture images of the scene 108 from a different perspective compared to other ones of the trackers 113. Accordingly, the trackers 113 and the cameras 112 can have different spectral sensitives (e.g., infrared vs. visible wavelength). In some embodiments, the trackers 113 capture image data of a plurality of optical markers (e.g., fiducial markers, marker balls) in the scene 108, such as markers 111 coupled to the instrument 101.

In the illustrated embodiment, the sensor array 110 further includes a depth sensor 114. In some embodiments, the depth sensor 114 includes (i) one or more projectors 116 that project a structured light pattern onto/into the scene 108 and (ii) one or more depth cameras 118 (which can also be referred to as second cameras) that capture second image data of the scene 108 including the structured light projected onto the scene 108 by the projector 116. The projector 116 can project a speckled pattern or a pattern of dots, for example. The projector 116 and the depth cameras 118 can operate in the same wavelength and, in some embodiments, can operate in a wavelength different than the cameras 112. For example, the cameras 112 can capture the first image data in the visible spectrum, while the depth cameras 118 capture the second image data in the infrared spectrum. In some embodiments, the depth cameras 118 have a resolution that is less than a resolution of the cameras 112. For example, the depth cameras 118 can have a resolution that is less than 70%, 60%, 50%, 40%, 30%, or 20% of the resolution of the cameras 112. In other embodiments, the depth sensor 114 can include other types of dedicated depth detection hardware (e.g., a LiDAR detector) for determining the surface geometry of the scene 108. In other embodiments, the sensor array 110 can omit the projector 116 and/or the depth cameras 118.

In the illustrated embodiment, the processing device 102 includes an image processing device 103 (e.g., an image processor, an image processing module, an image processing unit), a registration processing device 105 (e.g., a registration processor, a registration processing module, a registration processing unit), and a tracking processing device 107 (e.g., a tracking processor, a tracking processing module, a tracking processing unit). The image processing device 103 can (i) receive the first image data captured by the cameras 112 (e.g., light field images, light field image data, RGB images) and depth information from the depth sensor 114 (e.g., the second image data captured by the depth cameras 118), and (ii) process the image data and depth information to synthesize (e.g., generate, reconstruct, render) a three-dimensional (3D) output image of the scene 108 corresponding to a virtual camera perspective (e.g., a novel camera perspective). The output image can correspond to an approximation of an image of the scene 108 that would be captured by a camera placed at an arbitrary position and orientation corresponding to the virtual camera perspective. In some embodiments, the image processing device 103 can further receive and/or store calibration data for the cameras 112 and/or the depth cameras 118 and synthesize the output image based on the image data, the depth information, and/or the calibration data. More specifically, the depth information and the calibration data can be used/combined with the images from the cameras 112 to synthesize the output image as a 3D (or stereoscopic 2D) rendering of the scene 108 as viewed from the virtual camera perspective. In some embodiments, the image processing device 103 can synthesize the output image using any of the methods disclosed in U.S. patent application Ser. No. 16/457,780, filed Jun. 28, 2019, and titled “SYNTHESIZING AN IMAGE FROM A VIRTUAL PERSPECTIVE USING PIXELS FROM A PHYSICAL IMAGER ARRAY WEIGHTED BASED ON DEPTH ERROR SENSITIVITY,” which is incorporated herein by reference in its entirety. In other embodiments, the image processing device 103 can generate the virtual camera perspective based only on the images captured by the cameras 112—without utilizing depth information from the depth sensor 114. For example, the image processing device 103 can generate the virtual camera perspective by interpolating between the different images captured by one or more of the cameras 112. In some embodiments the image processing device 103 can utilize a neural radiance field (NeRF) rendering algorithm to synthesize and render an output image of the scene 108 based on RGB images captured by the cameras 112 and depth data captured by the depth sensor 114.

The image processing device 103 can synthesize the output image from images captured by a subset (e.g., two or more) of the cameras 112 in the sensor array 110, and does not necessarily utilize images from all of the cameras 112. For example, for a given virtual camera perspective, the processing device 102 can select a stereoscopic pair of images from two of the cameras 112. In some embodiments, such a stereoscopic pair can be selected to be positioned and oriented to most closely match the virtual camera perspective. In some embodiments, the image processing device 103 (and/or the depth sensor 114) estimates a depth for each surface point of the scene 108 relative to a common origin to generate a point cloud and/or a 3D mesh that represents the surface geometry of the scene 108. Such a representation of the surface geometry can be referred to as a surface reconstruction, a 3D reconstruction, a 3D surface reconstruction, a depth map, a depth surface, and/or the like. In some embodiments, the depth cameras 118 of the depth sensor 114 detect the structured light projected onto the scene 108 by the projector 116 to estimate depth information of the scene 108. In some embodiments, the image processing device 103 estimates depth from multiview image data from the cameras 112 using techniques such as light field correspondence, stereo block matching, photometric symmetry, correspondence, defocus, block matching, texture-assisted block matching, structured light, and the like, with or without utilizing information collected by the depth sensor 114. In other embodiments, depth may be acquired by a specialized set of the cameras 112 performing the aforementioned methods in another wavelength. In some embodiments, the image processing device 103 can generate a stereoscopic view by selecting images from a pair of the cameras 112 using any of the methods disclosed in U.S. patent application Ser. No. 17/521,235, filed Nov. 11, 2021, and titled “METHODS FOR GENERATING STEREOSCOPIC VIEWS IN MULTICAMERA SYSTEMS, AND ASSOCIATED DEVICES AND SYSTEMS,” which is incorporated herein by reference in its entirety.

In some embodiments, the registration processing device 105 receives and/or stores initial image data, such as image data of a three-dimensional volume of a patient (3D image data). The image data can include, for example, computerized tomography (CT) scan data, magnetic resonance imaging (MRI) scan data, ultrasound images, fluoroscope images, and/or other medical or other image data. The image data can be segmented or unsegmented. The registration processing device 105 can register the initial image data to the real-time images captured by the cameras 112 and/or the depth sensor 114 by, for example, determining one or more transforms/transformations/mappings between the two. The processing device 102 (e.g., the image processing device 103) can then apply the one or more transformations to the initial image data such that the initial image data can be aligned with (e.g., overlaid on) the output image of the scene 108 in real-time or near real-time on a frame-by-frame basis, even as the virtual perspective changes. That is, the image processing device 103 can fuse the initial image data with the real-time output image of the scene 108 to present a mediated-reality view that enables, for example, a surgeon to simultaneously view a surgical site in the scene 108 and the underlying 3D anatomy of a patient undergoing an operation. In some embodiments, the registration processing device 105 can register the initial image data to the real-time images by using any of the methods disclosed in U.S. patent application Ser. No. 17/140,885, filed Jan. 4, 2021, and titled “METHODS AND SYSTEMS FOR REGISTERING PREOPERATIVE IMAGE DATA TO INTRAOPERATIVE IMAGE DATA OF A SCENE, SUCH AS A SURGICAL SCENE,” and/or U.S. patent application Ser. No. 18/084,389, filed Dec. 19, 2022, and titled “METHODS AND SYSTEMS FOR REGISTERING PREOPERATIVE IMAGE DATA TO INTRAOPERATIVE IMAGE DATA OF A SCENE, SUCH AS A SURGICAL SCENE,” each of which is incorporated by reference herein in its entirety.

In some embodiments, the tracking processing device 107 processes positional data captured by the trackers 113 to track objects (e.g., the instrument 101) within the vicinity of the scene 108. For example, the tracking processing device 107 can determine the position of the markers 111 in the 2D images captured by two or more of the trackers 113, and can compute the 3D position of the markers 111 via triangulation of the 2D positional data. More specifically, in some embodiments the trackers 113 include dedicated processing hardware for determining positional data from captured images, such as a centroid of the markers 111 in the captured images. The trackers 113 can then transmit the positional data to the tracking processing device 107 for determining the 3D position of the markers 111. In other embodiments, the tracking processing device 107 can receive the raw image data from the trackers 113. In a surgical application, for example, the tracked object can comprise a surgical instrument, an implant, a hand or arm of a physician or assistant, and/or another object having the markers 111 mounted thereto. In some embodiments, the processing device 102 can recognize the tracked object as being separate from the scene 108, and can apply a visual effect to the 3D output image to distinguish the tracked object by, for example, highlighting the object, labeling the object, and/or applying a transparency to the object.

In some embodiments, functions attributed to the processing device 102, the image processing device 103, the registration processing device 105, and/or the tracking processing device 107 can be practically implemented by two or more physical devices. For example, in some embodiments a synchronization controller (not shown) controls images displayed by the projector 116 and sends synchronization signals to the cameras 112 to ensure synchronization between the cameras 112 and the projector 116 to enable fast, multi-frame, multicamera structured light scans. Additionally, such a synchronization controller can operate as a parameter server that stores hardware specific configurations such as parameters of the structured light scan, camera settings, and camera calibration data specific to the camera configuration of the sensor array 110. The synchronization controller can be implemented in a separate physical device from a display controller that controls the display device 104, or the devices can be integrated together.

The processing device 102 can comprise a processor and a non-transitory computer-readable storage medium that stores instructions that when executed by the processor, carry out the functions attributed to the processing device 102 as described herein. Although not required, aspects and embodiments of the present technology can be described in the general context of computer-executable instructions, such as routines executed by a general-purpose computer, e.g., a server or personal computer. Those skilled in the relevant art will appreciate that the present technology can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers and the like. The present technology can be embodied in a special purpose computer or data processor that is specifically programmed, configured or constructed to perform one or more of the computer-executable instructions explained in detail below. Indeed, the term “computer” (and like terms), as used generally herein, refers to any of the above devices, as well as any data processor or any device capable of communicating with a network, including consumer electronic goods such as game devices, cameras, or other electronic devices having a processor and other components, e.g., network communication circuitry.

The present technology can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or sub-routines can be located in both local and remote memory storage devices. Aspects of the present technology described below can be stored or distributed on computer-readable media, including magnetic and optically readable and removable computer discs, stored as in chips (e.g., EEPROM or flash memory chips). Alternatively, aspects of the present technology can be distributed electronically over the Internet or over other networks (including wireless networks). Those skilled in the relevant art will recognize that portions of the present technology can reside on a server computer, while corresponding portions reside on a client computer. Data structures and transmission of data particular to aspects of the present technology are also encompassed within the scope of the present technology.

The virtual camera perspective is controlled by an input controller 106 that can update the virtual camera perspective based on user driven changes to the camera's position and rotation. The output images corresponding to the virtual camera perspective can be outputted to the display device 104. In some embodiments, the image processing device 103 can vary the perspective, the depth of field (e.g., aperture), the focus plane, and/or another parameter of the virtual camera (e.g., based on an input from the input controller) to generate different 3D output images without physically moving the sensor array 110. The display device 104 can receive output images (e.g., the synthesized 3D rendering of the scene 108) and display the output images for viewing by one or more viewers. In some embodiments, the processing device 102 receives and processes inputs from the input controller 106 and processes the captured images from the sensor array 110 to generate output images corresponding to the virtual perspective in substantially real-time or near real-time as perceived by a viewer of the display device 104 (e.g., at least as fast as the frame rate of the sensor array 110).

Additionally, the display device 104 can display a graphical representation on/in the image of the virtual perspective of any (i) tracked objects within the scene 108 (e.g., a surgical instrument) and/or (ii) registered or unregistered initial image data. That is, for example, the system 100 (e.g., via the display device 104) can blend augmented data into the scene 108 by overlaying and aligning information on top of “passthrough” images of the scene 108 captured by the cameras 112 and/or generated by images captured by the cameras 112. Moreover, the system 100 can create a mediated-reality experience where the scene 108 is reconstructed using light field image data of the scene 108 captured by the cameras 112, and where instruments are virtually represented in the reconstructed scene via information from the trackers 113. Additionally or alternatively, the system 100 can remove the original scene 108 and completely replace it with a registered and representative arrangement of the initial image data, thereby removing information in the scene 108 that is not pertinent to a user's task.

The display device 104 can comprise, for example, a head-mounted display device, a monitor, a computer display, and/or another display device. In some embodiments, the input controller 106 and the display device 104 are integrated into a head-mounted display device and the input controller 106 comprises a motion sensor that detects position and orientation of the head-mounted display device. In some embodiments, the system 100 can further include a separate tracking system (not shown), such an optical tracking system, for tracking the display device 104, the instrument 101, and/or other components within the scene 108. Such a tracking system can detect a position of the head-mounted display device 104 and input the position to the input controller 106. The virtual camera perspective can then be derived to correspond to the position and orientation of the head-mounted display device 104 in the same reference frame and at the calculated depth (e.g., as calculated by the depth sensor 114) such that the virtual perspective corresponds to a perspective that would be seen by a viewer wearing the head-mounted display device 104. Thus, in such embodiments the head-mounted display device 104 can provide a real-time rendering of the scene 108 as it would be seen by an observer without the head-mounted display device 104. Alternatively, the input controller 106 can comprise a user-controlled control device (e.g., a mouse, pointing device, handheld controller, gesture recognition controller) that enables a viewer to manually control the virtual perspective displayed by the display device 104.

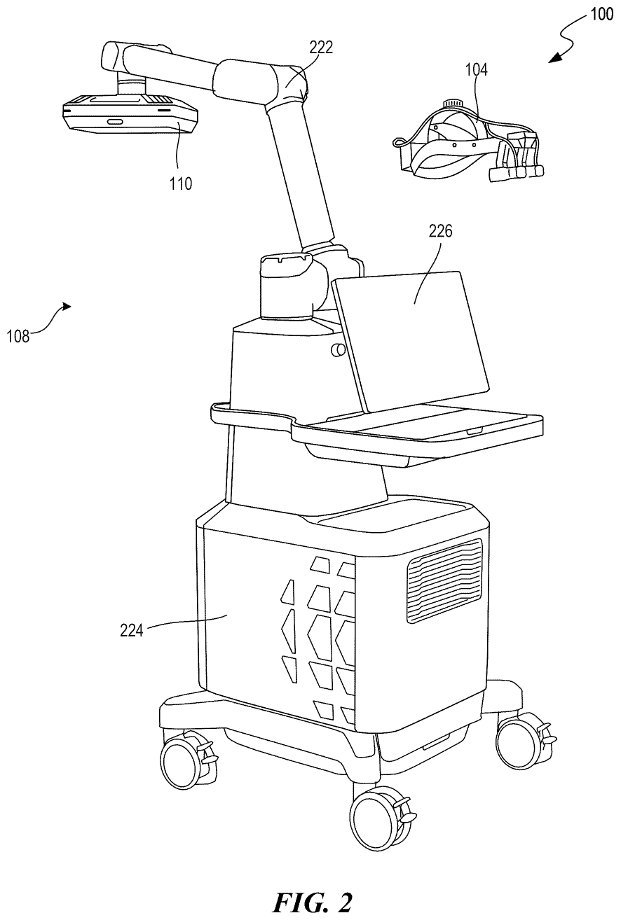

FIG. 2 is a perspective view of an environment (e.g., a surgical environment) employing the system 100 (e.g., for a surgical application) in accordance with embodiments of the present technology. In the illustrated embodiment, the sensor array 110 is positioned over the scene 108 (e.g., a surgical site) and supported/positioned via a mover 222 that is operably coupled to a workstation 224. In some embodiments, the mover 222 is manually movable to position the sensor array 110 while, in other embodiments, the mover 222 is robotically controlled in response to the input controller 106 (FIG. 1) and/or another controller. Accordingly, the mover 222 can be referred to as a robotic mover, a robotic arm, a robotically-controlled arm, and/or the like. The mover 222 allows the sensor array 110 to be precisely moved relative to the scene 108 such that the sensor array 110 is mobile relative to the scene 108.

In the illustrated embodiment, the display device 104 is a head-mounted display device (e.g., a virtual reality headset, augmented reality headset). The workstation 224 can include a computer to control various functions of the processing device 102, the display device 104, the input controller 106, the sensor array 110, and/or other components of the system 100 shown in FIG. 1. Accordingly, in some embodiments the processing device 102 and the input controller 106 are each integrated in the workstation 224. In some embodiments, the workstation 224 includes a secondary display 226 that can display a user interface for performing various configuration functions, a mirrored image of the display on the display device 104, and/or other useful visual images/indications. In other embodiments, the system 100 can include more or fewer display devices. For example, in addition to (or alternatively to) the display device 104 and the secondary display 226, the system 100 can include another display (e.g., a medical grade computer monitor) visible to the user wearing the display device 104.

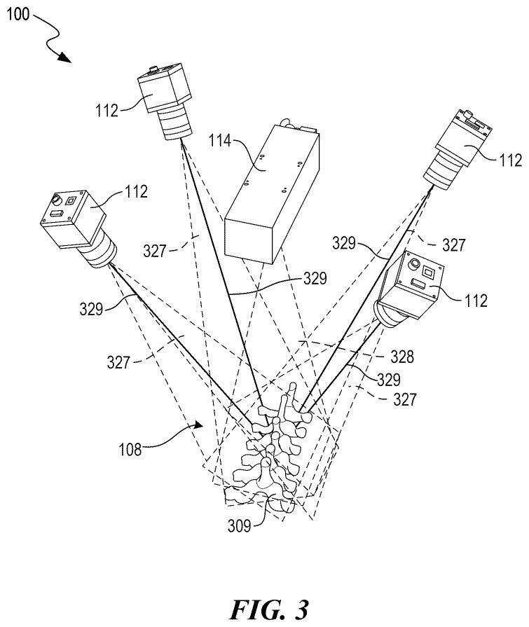

FIG. 3 is an isometric view of a portion of the system 100 illustrating four of the cameras 112 in accordance with embodiments of the present technology. Other components of the system 100 (e.g., other portions of the sensor array 110, the processing device 102, etc.) are not shown in FIG. 3 for the sake of clarity. In the illustrated embodiment, each of the cameras 112 has a field of view 327 and a focal axes 329. Likewise, the depth sensor 114 can have a field of view 328 aligned with a portion of the scene 108. The cameras 112 can be oriented such that the fields of view 327 are aligned with a portion of the scene 108 and at least partially overlap one another to together define an imaging volume. In some embodiments, some or all of the field of views 327, 328 at least partially overlap. For example, in the illustrated embodiment the fields of view 327, 328 converge toward a common measurement volume including a portion of a spine 309 of a patient (e.g., a human patient) located in/at the scene 108. In some embodiments, the cameras 112 are further oriented such that the focal axes 329 converge to a common point in the scene 108. In some aspects of the present technology, the convergence/alignment of the focal axes 329 can generally maximize disparity measurements between the cameras 112. In some embodiments, the cameras 112 and the depth sensor 114 are fixedly positioned relative to one another (e.g., rigidly mounted to a common frame) such that a relative positioning of the cameras 112 and the depth sensor 114 relative to one another is known and/or can be readily determined via a calibration process. In other embodiments, the system 100 can include a different number of the cameras 112 and/or the cameras 112 can be positioned differently relative to another.

Referring to FIGS. 1-3 together, in some aspects of the present technology the system 100 can generate a digitized view of the scene 108 that provides a user (e.g., a surgeon) with increased “volumetric intelligence” of the scene 108. For example, the digitized scene 108 can be presented to the user from the perspective, orientation, and/or viewpoint of their eyes such that they effectively view the scene 108 as though they were not viewing the digitized image (e.g., as though they were not wearing the head-mounted display device 104). However, the digitized scene 108 permits the user to digitally rotate, zoom, crop, or otherwise enhance their view to, for example, facilitate a surgical workflow. Likewise, initial image data, such as CT scans and/or MRI data, can be registered to and overlaid over the image of the scene 108 to allow a surgeon to view these data sets together. Such a fused view can allow the surgeon to visualize aspects of a surgical site that may be obscured in the physical scene 108—such as regions of bone and/or tissue that have not been surgically exposed.

### II. Selected Embodiments of Selecting and Managing Preoperative and Intraoperative Image Data of Imaging Systems

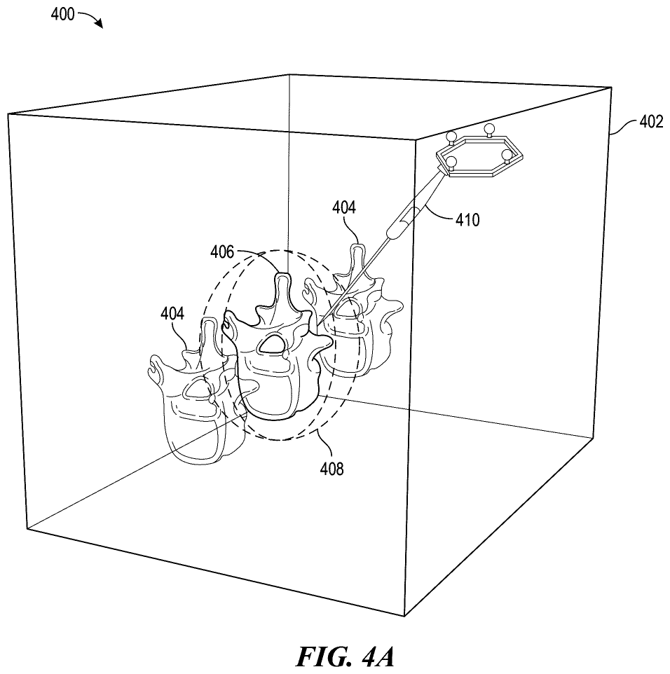

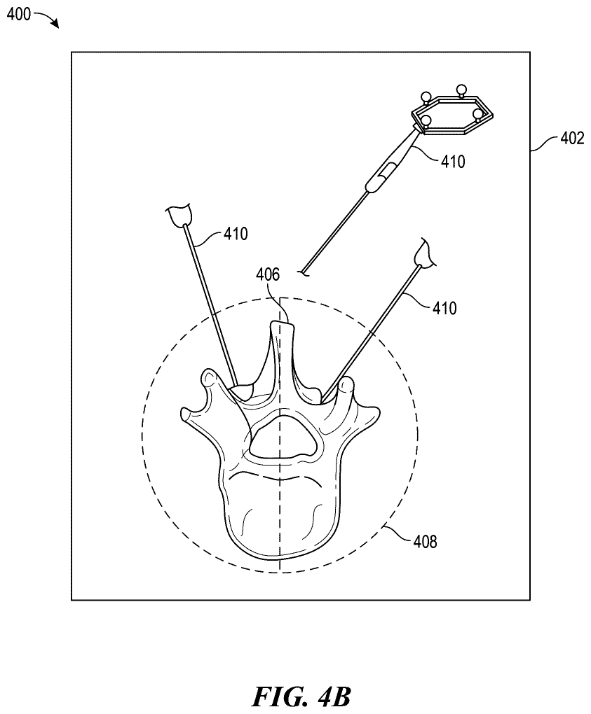

FIGS. 4A and 4B are an isometric view and a cross-sectional view, respectively, of a tracking environment 400 imaged/sensed by the system 100 of FIG. 1 in accordance with embodiments of the present technology. Referring to FIGS. 4A and 4B, the environment 400 includes a tracking region 402 (e.g., a tracking volume) and one or more physical objects within the tracking region 402. In the illustrated embodiment, the physical object comprises a spine including a reference or selected vertebra 406 (e.g., a selected object) and adjacent, non-selected vertebrae 404 (e.g., non-selected objects). While illustrated in the context of a spine including discrete vertebrae 404, 406, in other embodiments the object can comprise other types of bone (e.g., leg bones, arm bones, portions of a skull, etc.) and/or other physical objects within a scene. One or more instruments 410 can be selectively positioned within and moved through the tracking region 402. The instrument 410 can be the same as or similar to the instrument 101 illustrated and described in detail above with reference to FIG. 1. In some embodiments, a bounding region 408 (shown schematically) is defined by the system 100 around the selected vertebra 406. Embodiments of the environment 400 can include different and/or additional components and/or can be connected in different ways.

The tracking region 402 is a three-dimensional space within which the system 100 (FIG. 1) can monitor and track the position and movement of objects, such as surgical instruments (e.g., the instrument 410) and/or anatomical or other structures (e.g., the vertebrae 404, 406), and provide real-time updates on the objects' locations and/or movements. The tracking region 402 can provide the spatial context for the interactions between the instruments and the anatomical structures during a surgical procedure. The tracking region 402 can encompass all or a portion of the scene 108 (FIG. 1) within which the system 100 operates and can comprise a volume in which the system 100 can track relevant objects (e.g., objects used in a surgical procedure), such as the vertebrae 404, 406 and/or the instrument 410. For example, with reference to FIG. 1, the tracking region 402 can encompass a volume imaged by the trackers 113, such as a volume imaged by two or more (e.g., all) of the trackers 113 within the scene 108. While illustrated as a rectangular solid or cube, the tracking region 402 can have any suitable volumetric shape. Once the spatial boundaries are established for the tracking region 402, the system 100 can be calibrated to recognize and differentiate between the different surgical instruments and/or different anatomical structures therein. For example, the system 100 can register the objects, such as the instrument 410 and/or the vertebrae 404, 406, within the system 100 by assigning the objects unique identifiers and defining the objects' geometrical parameters (e.g., using the registration processing device 105 of FIG. 1).

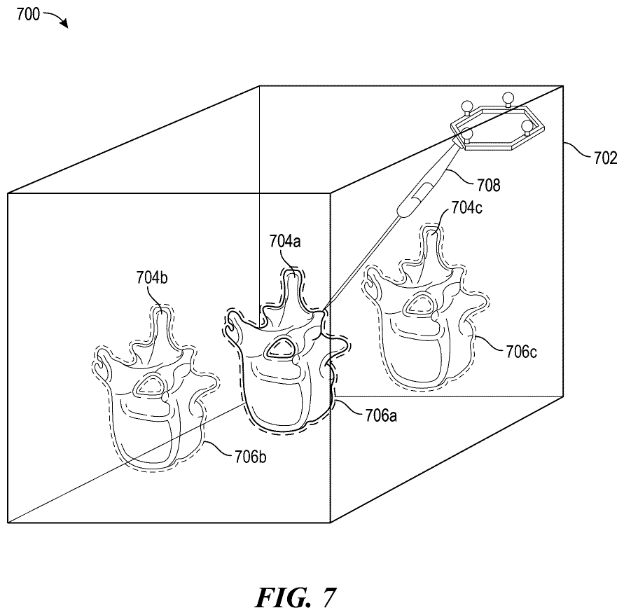

The non-selected vertebrae 404 can comprise the vertebrae located near and/or adjacent to the selected vertebra 406 (e.g., one or more adjacent vertebral levels) within the tracking region 402. The non-selected vertebrae 404 can provide context and reference points for positioning and navigation. For example, information about the non-selected vertebrae 404 can aid the system 100 and/or the users of the system 100 to identify the relative position of the selected vertebra 406 and/or the instrument 410. The selected vertebra 406 is the specific vertebral (or other) bone that is the primary object for display by the system 100, as described in further detail below with reference to FIGS. 7-9B. The selected vertebra 406, for example, can be a vertebra being operated on and/or examined during a surgical procedure. The selected vertebra 406 is the main reference point for the surgical instruments and the bounding region 408 to allow for targeted and precise surgical interventions. The selected vertebra 406 is the primary reference point within the tracking region 402 and can be selected as described in further detail below with reference to FIGS. 7-9B.

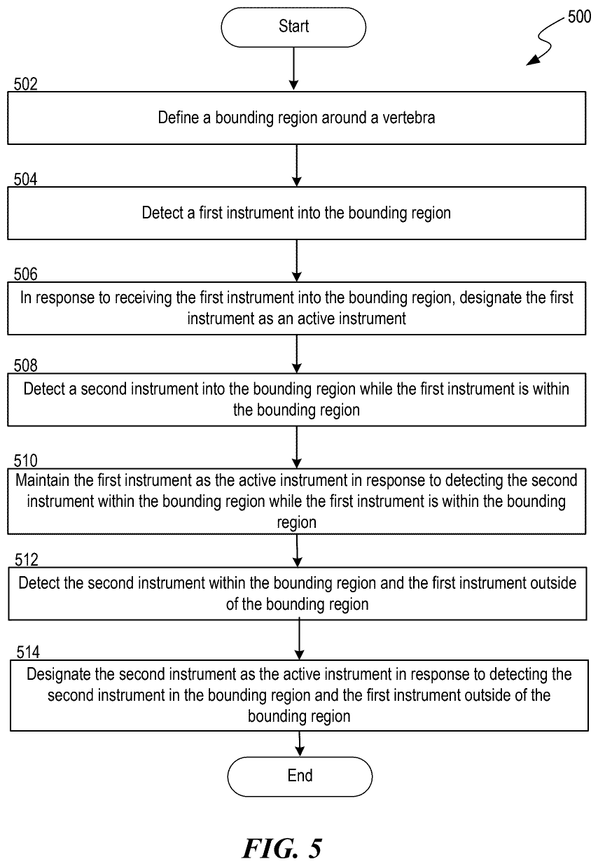

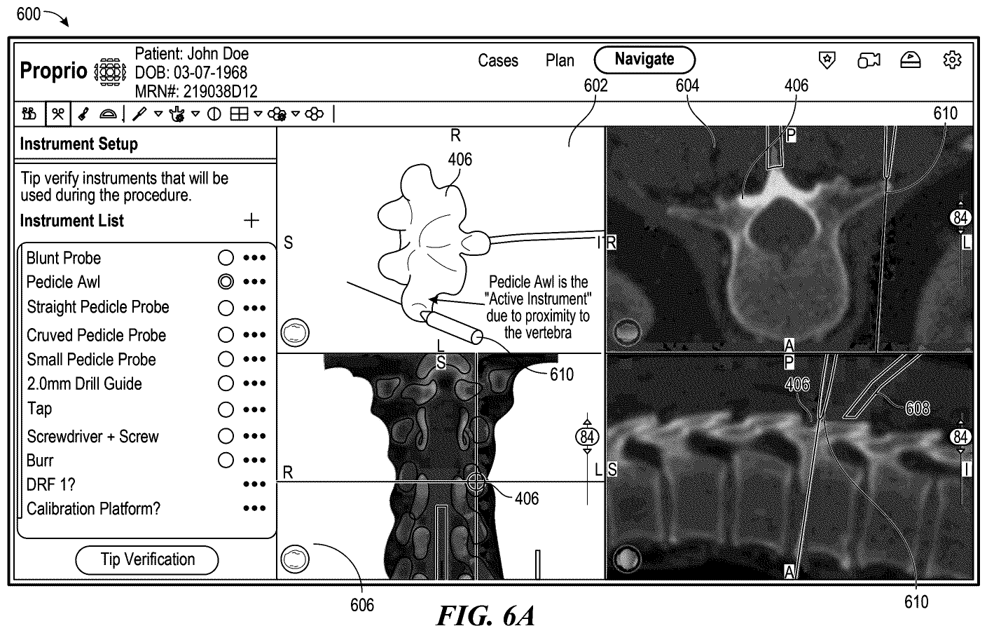

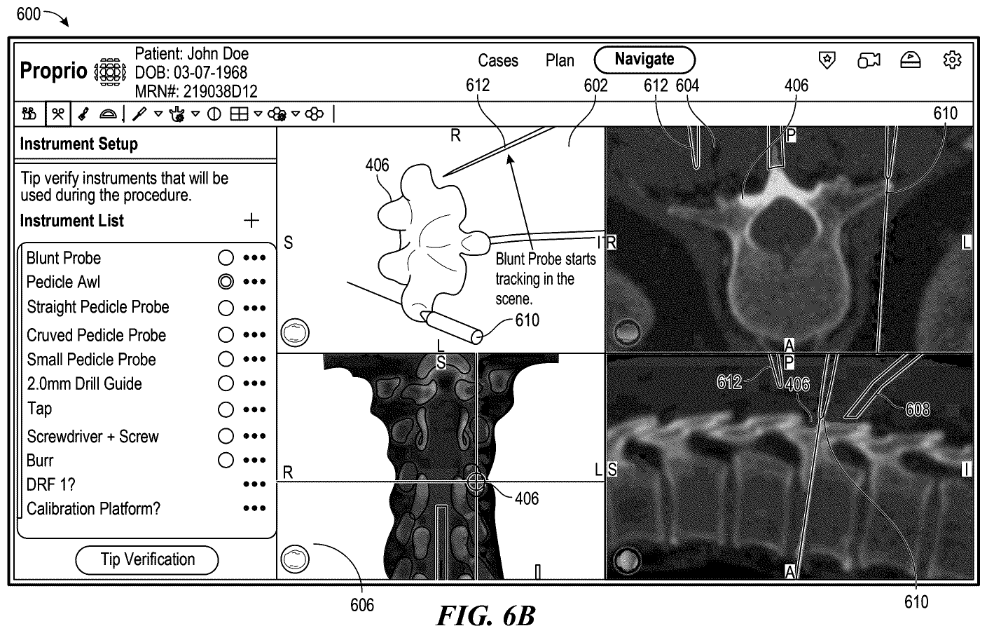

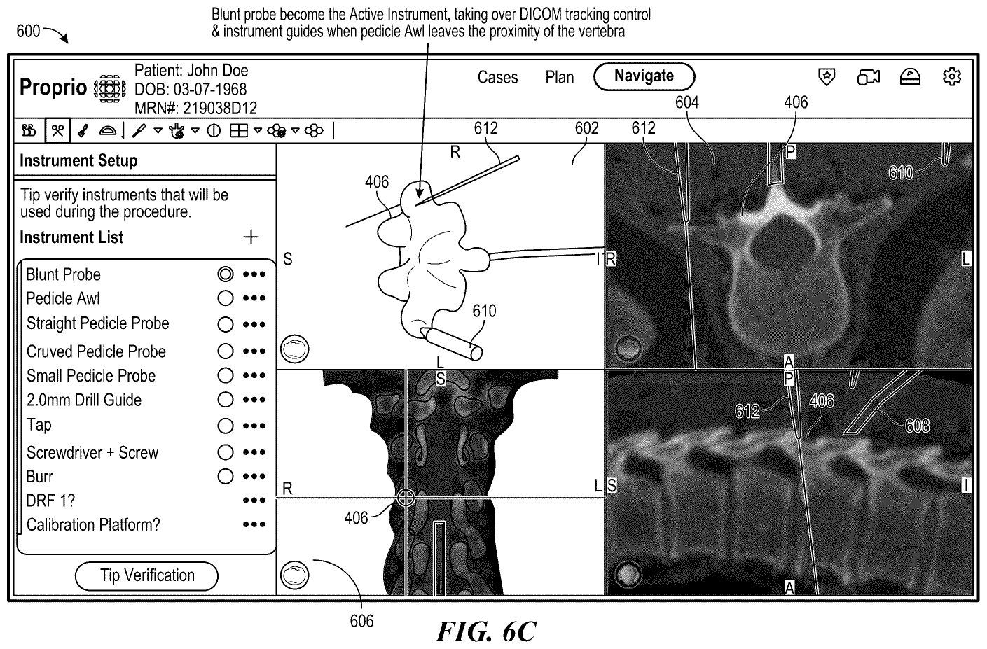

The bounding region 408 is a defined volume around the selected vertebra 406 which the system 100 can utilize to determine an active instrument, as described in further detail below with reference to FIGS. 5-6C. The active instrument can control the display of information on a display of the system 100 (FIG. 1). The bounding region 408 can be defined by pre-defined shapes such as spheres, boxes, or custom mesh surfaces that conform to the vertebra's geometry. The bounding region 408 is a spatial boundary that is used to monitor and track surgical instruments entering the area. The bounding region 408 allows the system to dynamically update the designation of an active instrument, which is described in further detail below with reference to FIGS. 5-6C. The instrument 410 can be the surgical tool or device being used (e.g., a surgical tool, a surgical implant, a surgical tool coupled to a surgical instrument) within the tracking region 402. The interactions of the instrument 410 with the selected vertebra 406 and the bounding region 408 can be used by the system to designate the active instrument.

When a surgical procedure begins, the system 100 can identify the selected vertebra 406 using predetermined settings, or identify the selected vertebra 406 dynamically. Methods of identifying the selected vertebra 406 dynamically are discussed in further detail below with reference to FIGS. 7-9B. As the instrument 410 approaches, enters, and moves through the bounding region 408, the system 100 can continuously track the position and movement of the instrument 410. Methods of tracking the instrument 410 are described in detail below with reference to FIG. 5. The system 100 uses the proximity threshold of the bounding region 408 to automatically designate the instrument 410 as active or non-active, as described in further detail below with reference to FIG. 5.

FIG. 5 is a flow diagram of a process or method 500 for dynamically selecting and managing an active instrument in the tracking environment 400 of FIGS. 4A and 4B in accordance with embodiments of the present technology. Although some features of the method 500 are described in the context of the system 100 shown in FIGS. 1-4B for the sake of illustration, one skilled in the art will readily understand that the method 500 can be carried out using other suitable systems and/or devices described herein. Similarly, while reference is made herein to preoperative image data, intraoperative image data, and a surgical scene, the method 500 can be used with other types of information about other scenes (e.g., non-surgical scenes). Likewise, implementations and embodiments can include different and/or additional acts or can perform the acts in different orders.

At block 502, the method 500 can include defining a bounding region (e.g., the bounding region 408 of FIGS. 4A and 4B) around a vertebra (e.g., the selected vertebra 406 of FIGS. 4A and 4B). The bounding region 408 can be defined by setting specific predefined parameters within the system. For example, predefined parameters can define the shape and size of the bounding region 408, which can be adjusted to fit the anatomical shape of the vertebra. Shapes for the bounding region 408 can vary, and can include spheres, boxes, and/or custom mesh surfaces that match the vertebra's geometry (e.g., a bounding region that extends a predefined distance from the anatomical surface of the selected vertebra). In some embodiments, the bounding region 408 can be dynamically determined using imaging data from MRI, CT, and/or scans. The imaging data can be processed to create a 3D model (e.g., a 3D mesh) of the surface geometry of the vertebra and/or the tissue surrounding the vertebra, and define a bounding region that corresponds to the anatomical contours.

More specifically, FIGS. 6A-6C illustrate a user interface 600 (e.g., a display) visible to a user of the system via the display device 104 (e.g., a head-mounted display device) and/or the secondary display 226 in accordance with embodiments of the present technology. FIGS. 6A-6C illustrate operation of the user interface 600 according to the method 500 of FIG. 5. Referring to FIGS. 6A-6C, in the illustrated embodiment the user interface 600 displays previously-captured data corresponding to the vertebrae 404, 406 and registered to the physical object in the scene. More specifically, the user interface 600 can include a primary viewport 602 or panel displaying a 3D view of the selected vertebra 406 and secondary panels or viewports 604, 606, 608 (individually identified as first, second, and third secondary panels or viewports 604, 606, 608, respectively) each displaying a corresponding different 2D view (e.g., a coronal, sagittal, and/or axial 2D view) of the selected vertebra 406. The previously-captured image data from which the 2D and 3D views are generated can be preoperative image data. For example, in the illustrated embodiment the 3D image data of the selected vertebra 406 displayed in the primary viewport 602 includes 3D geometric and/or volumetric data of the selected vertebra 406, such as CT scan data, MRI scan data, ultrasound image data, fluoroscopic image data, and/or other medical or other image data. In some embodiments, the previously-captured image data can be captured intraoperatively. For example, the previously-captured image data can comprise 2D or 3D X-ray images, fluoroscopic images, CT images, MRI images, etc., and combinations thereof, captured of the patient within an operating room. In some embodiments, the previously-captured image data comprises a point cloud, three-dimensional (3D) mesh, and/or another 3D data set. In some embodiments, the previously-captured image data comprises segmented 3D CT scan data or 2D slice data of some or all of the spine of the patient (e.g., segmented on a per-vertebra basis). The method 500 can include defining the bounding region around the selected vertebra 406 in two or three dimensions.

At block 504, the method 500 can include detecting that a first instrument (e.g., the instrument 410 of FIGS. 4A and 4B) has entered into the bounding region (e.g., the bounding region 408 of FIGS. 4A and 4B). When the first instrument 610 moves into the bounding region 408, the system detects this movement based on the predefined parameters of the bounding region and the tracked position of the instrument.

To ensure that the system accurately detects when surgical instruments enter or exit the bounding region 408, proximity thresholds can be defined. The proximity thresholds are predefined distances from the edges of the bounding region 408 that trigger the system to recognize and classify an instrument as entering the bounding region 408. In some embodiments, the proximity threshold is zero (e.g., the bounding region 408 is the proximity threshold). When an instrument passes a proximity threshold, the system can automatically update a data visualization accordingly.

In some embodiments, the definition of the bounding region 408 can be further refined based on preoperative imaging data. For instance, 3D scans of the patient's spine can be used to create models of each vertebra, which can then be used to generate the bounding region 408 of the vertebra. The integration of imaging data allows for even greater precision, as the bounding region 408 can be tailored to the specific anatomy of the patient. Additionally, in some embodiments, the system can incorporate real-time adjustments to modify the bounding region 408 as needed during the procedure to account for any changes in the positioning or orientation of the vertebrae.

For example, FIG. 6A displays an overlaid representation of a tracked first instrument 610, and illustrate the display of the 3D and 2D image data as the tracked first instrument 610, approaches, enters, moves through, and/or exits a bounding region (e.g., the bounding region 408 of FIGS. 4A and 4B) around the selected vertebra 406 in accordance with embodiments of the present technology. In the illustrated embodiment, the 3D image data displayed in the primary viewport 602 includes volumetric data of a patient's spine including a vertebra (e.g., the selected vertebra 406). Further, the first instrument 610 is shown as inserted into the selected vertebra 406 during a procedure. In some embodiments, the first instrument 610 can be a pedicle awl (as depicted in FIG. 6A), a probe (as depicted by a blunt probe that is a second instrument 612 in FIG. 6B), a drill, a screw, and/or another tool used during a procedure to implant a screw or other implantable device in the vertebra.

The system can detect the movement using, for example, the camera array 110 that includes cameras 112 and trackers 113 in FIG. 1. In some embodiments, the system can detect the movement, using various technologies such as optical tracking with optical markers (e.g., markers 111), electromagnetic tracking, and/or infrared tracking. Methods of optical tracking are discussed further with reference to FIG. 1. Electromagnetic tracking uses a magnetic field to detect the position and orientation of a surgical instrument equipped with electromagnetic sensors. As the instrument moves, the sensors can detect changes in the magnetic field, and the system processes the changes to calculate the instrument's position and orientation in real time. Infrared tracking uses infrared light to detect and track the position of markers or sensors on surgical instruments. Infrared markers or LEDs are placed on the instruments, and an infrared light source illuminates the area, or the markers themselves emit infrared light. Infrared cameras capture the reflected or emitted infrared light from the markers. The system processes the images to identify the markers and calculate their positions, updating them continuously to track the instrument in real time or near real time.

The system can provide feedback in response to detecting the first instrument 610 by indicating the entry of the first instrument 610 into the bounding region 408. Once the first instrument 610 is detected within the bounding region 408, the system can highlight the position of the first instrument 610 on preoperative images of the anatomy, provide real-time metrics related to the first instrument 610, and/or trigger specific software functionalities such as zooming in on the area associated with the first instrument 610 or overlaying relevant data.

At block 506, in response to detecting that the first instrument (e.g., the first instrument 610 in FIGS. 6A-6C) has entered into the bounding region (e.g., the bounding region 408 of FIGS. 4A and 4B), the method 500 can include designating the first instrument 610 as an active instrument. The designation occurs automatically when the system identifies that the first instrument 610 has crossed the predefined boundary surrounding the target area, such as a specific vertebra. The active instrument affects what previously-captured image data is shown, such as image data of a three-dimensional volume of a patient (3D image data). The system dynamically updates the displayed 3D image data based on the position and interaction of the active instrument. For instance, as the instrument moves closer to or interacts with specific anatomical structures, the system can highlight the corresponding areas, adjust the viewing angles, and/or provide cross-sectional views in accordance with the instrument's location. Referring to FIG. 6A, for example, the first instrument 610 is designated as the active instrument due to the proximity of the first instrument 610 with the selected vertebra 406 (e.g., the first instrument 610 is within the proximity threshold of the bounding region 408 of the selected vertebra 406). The position of the active instrument 610 can be used to, for example, change the 2D views displayed in the secondary viewports 604, 606, 608 (e.g., a slice corresponding to the position and/or orientation of a tip and/or other portion of the first instrument 610). That is, the 2D images shown in the secondary viewports 604, 606, 608 can be updated to correspond to 2D slices of the three-dimensional image data in the primary viewport 602 taken along a plane corresponding to the position (e.g., tip) of the first instrument 610, and can be dynamically updated in real time or near real time as the first instrument 610 moves relative to the selected vertebra 406.

At block 508, the method 500 can include detecting that a second instrument (e.g., the second instrument 612 in FIGS. 6B and 6C) has entered into the bounding region (e.g., the bounding region 408 of FIGS. 4A and 4B) while the first instrument (e.g., the first instrument 610 in FIGS. 6A-6C) is within the bounding region. The system 100 continues to monitor the spatial relationships and movements of both instruments. The detection of the second instrument 612 triggers a decision-making process within the system 100, which potentially leads to adjustments in the displayed data and/or the designation of a new active instrument if certain criteria (e.g., block 510 and/or block 512) are met. Methods of detecting that the second instrument 612 has entered into the bounding region are the same as or similar to methods of detecting that the first instrument 610 has entered in the bounding region at block 504.

Referring to FIG. 6B, for example, the second instrument 612 has entered within the proximity threshold of the bounding region 408 (e.g., FIGS. 4A and 4B) while the first instrument 610 remains within the bounding region 408. As described in greater detail below, in this scenario the active instrument status only applies to a single instrument (e.g., the first instrument 610 in FIG. 6A). For example, the system can detect that the first instrument 610 has entered into the bounding region 408 (FIGS. 4A and 4B) around the selected vertebra 406 before the second instrument 612 (block 504) and remains within the bounding region 408 such that the first instrument 610 remains designated as the active instrument. The display of the second instrument 612 in the image displays in the viewports 602, 604, 606, 608, despite the second instrument 612 not being designated as the active instrument, ensures that the viewer of the imaging system 100 is aware of all relevant instruments approaching the area of interest (e.g., scene 108). The early detection and display allows the viewer of the imaging system 100 to prepare for and visualize the instrument's potential interactions with the anatomical structures and other instruments within the surgical field. By displaying the second instrument 612 when the second instrument 612 enters the tracking region 402, the system 100 provides a comprehensive view of the surgical environment.

Referring to FIGS. 4A, 4B, and 6A, the second instrument 612 has yet to enter the proximity threshold of the bounding region 408 of the selected vertebra 406, and also has yet to enter the tracking region 402 of the selected vertebra 406. Since the second instrument 612 is outside both the bounding region 408 and the tracking region 402, the second instrument 612 remains undetected and is not displayed in the image data on the user interface 600. The tracking region 402 represents the broader volume within which the system 100 can monitor instruments, while the bounding region 408 is a more focused volume around the selected vertebra 406. The system 100 uses the tracking region 402 and the bounding region 408 to manage and prioritize the display of instruments, ensuring that only instruments within relevant proximity thresholds appear in the imaging interface. The approach helps to avoid cluttering the display with irrelevant data. As the second instrument 612 has not yet breached these thresholds, the second instrument 612 remains hidden from the image displays in the viewports 602, 604, 606, 608 until the second instrument 612 moves closer to the selected vertebra 406.

At block 510, the method 500 can include maintaining the first instrument (e.g., the first instrument 610 in FIGS. 6A-6C) as the active instrument in response to detecting that the second instrument (e.g., the second instrument 612 in FIGS. 6A-6C) has entered into the bounding region (e.g., the bounding region 408 of FIGS. 4A and 4B) while the first instrument 610 is within the bounding region. By maintaining the first instrument 610 as the active instrument, the system 100 continues to display pertinent data related to the position and/or orientation of the first instrument 610, and any interactions with the anatomical structures captured in the image data, in the viewports 602, 604, 606, 608. For example, the 2D slices displayed in the secondary viewports 604, 606, 608 can continue to be dynamically updated based on the position and/or orientation of the active first instrument 610. Maintaining the first instrument 610 as the active instrument decreases potential disruptions that can occur if the active instrument designation were to switch prematurely as additional instruments enter the tracking region 402, thereby allowing the viewer of the imaging system 100 to focus on the task at hand with a stable visual and data reference (e.g., 3D image data). The second instrument 612, although detected and tracked by the system 100, does not override the primary focus as the active instrument unless the first instrument 610 exits the bounding region 408.

At block 512, the method 500 can include detecting that the second instrument (e.g., the second instrument 612 in FIGS. 6A-6C) is within the bounding region (e.g., the bounding region 408 of FIGS. 4A and 4B) and that the first instrument (e.g., the first instrument 610 in FIGS. 6A-6C) is outside of the bounding region (e.g., when the tracking data indicates that the first instrument's 610 coordinates have moved beyond the limits of the bounding region). For example, during a surgical procedure, instruments may move in and out of specific target areas within the patient's anatomy. The detection of the second instrument 612 within the bounding region 408 while the first instrument 610 has been removed from the bounding region 408 triggers a transition in the system's 100 active instrument designation.

For example, FIG. 6C illustrates the second instrument 612 contacting an entry point on the selected vertebra 406, and thus reaching the proximity threshold of the bounding region 408 of the selected vertebra 406. In the illustrated embodiment, after contacting the selected vertebra 406 with the second instrument 612, the first instrument 610 exits the proximity threshold of the bounding region 408 of the selected vertebra 406, so the user interface 600 can display the 3D image data based on the second instrument 612 rather than the first instrument 610. For example, the user interface 600 can update and display the second instrument's position, orientation, and interactions with the selected vertebra 406. The second instrument 612 becomes the active instrument since the previously designated active instrument (e.g., first instrument 610) exits the bounding region 408. In FIG. 6C, if the first instrument 610 remained within the bounding region 408, the first instrument 610 would continue to be designated as the active instrument, and the user interface 600 would continue displaying the 3D image data based on the first instrument 610.

For example, at block 514, the method 500 can include designating the second instrument (e.g., the second instrument 612 in FIGS. 6A-6C) as the active instrument in response to detecting that the second instrument 612 is within the bounding region (e.g., the bounding region 408 of FIGS. 4A and 4B) and the first instrument (e.g., the first instrument 610 in FIGS. 6A-6C) is outside of the bounding region (e.g., has been removed from the bounding region). Specifically, since the first instrument 610 has exited the defined boundary, the system 100 updates the system's 100 designated active instrument to reflect the presence and actions of the newly detected second instrument 612. The dynamic adjustment ensures that the viewer of the imaging system 100 receives real time or near real time updates and visual feedback relevant to the current primary instrument interacting with the targeted anatomical structures. The system 100 maintains the continuity in surgical workflows and ensures that the displayed 3D image data and instrument interactions remain aligned and actionable throughout the procedure. The hierarchical approach (e.g., king of the hill approach) to instrument management ensures that the most relevant tool remains in focus until a clear and deliberate change is detected.

FIG. 7 is an isometric view of a tracking environment 700 imaged/sensed by the system 100 of FIG. 1 in accordance with embodiments of the present technology. The environment 700 includes a tracking region 702 (e.g., a tracking volume) and one or more physical objects within the tracking region 702. In the illustrated embodiment, the physical object comprises a spine including vertebrae 704 (e.g., objects; including individually identified first through third vertebra 704a-704c, respectively). In the illustrated embodiment, the second and third vertebrae 704b-c are adjacent to the first vertebra 704a. While illustrated in the context of a spine including discrete vertebrae 704, in other embodiments, the physical object can comprise other types of bone (e.g., leg bones, arm bones, portions of a skull, etc.) and/or other physical objects. The tracking region 702 can be the same as or similar to the tracking region 402 illustrated and described in detail above with reference to FIGS. 4A-6C. One or more instruments 708 can be selectively positioned within and moved through the tracking region 702. In some embodiments, bounding regions 706 (shown schematically; including individually identified first through third regions 706a-706c, respectively) are defined by the system 100 around each of the vertebrae 704. The bounding regions 706 can be the same as or similar to bounding region 408 illustrated and described in detail above with reference to FIGS. 4A-5. The instrument 708 can be the same as or similar to the instrument 101 illustrated and described in detail above with reference to FIG. 1. Embodiments of environment 700 can include different and/or additional components and/or can be connected in different ways.

The bounding region 706 associated with each of the vertebrae 704 can be a defined space that facilitates the automatic selection of a reference object—in FIG. 7, one of the vertebrae 704—based on the proximity of a tracked object, such as the surgical instrument 708. In some embodiments, the bounding regions 706 are tailored to match the anatomical contours of the corresponding vertebrae 704 using shapes such as spheres, boxes, custom mesh surfaces, and so forth (as described in detail above with reference to FIG. 5).

When the instrument 708 enters one of the bounding regions 706, the instrument 708 triggers the system 100 to recognize and designate the associated vertebra as an active vertebra. In some embodiments, the instrument 708 is an active instrument. Methods of determining the active instrument are discussed in detail above with reference to FIGS. 4A-6C. That is, in some embodiments only an instrument designated as an active instrument may trigger the designation of an active vertebra. For instance, in the illustrated embodiment the first vertebra 704a is designated as the active vertebra (e.g., as shown by darker shading in FIG. 7). However, if the instrument 708 enters the second bounding region 706b around the second vertebra 704b, the system 100 can automatically highlight the second vertebra 704b, designate the second vertebra 704b as the active vertebra, and/or use the second vertebra 704b for further actions such as displaying image data on a display of the system.

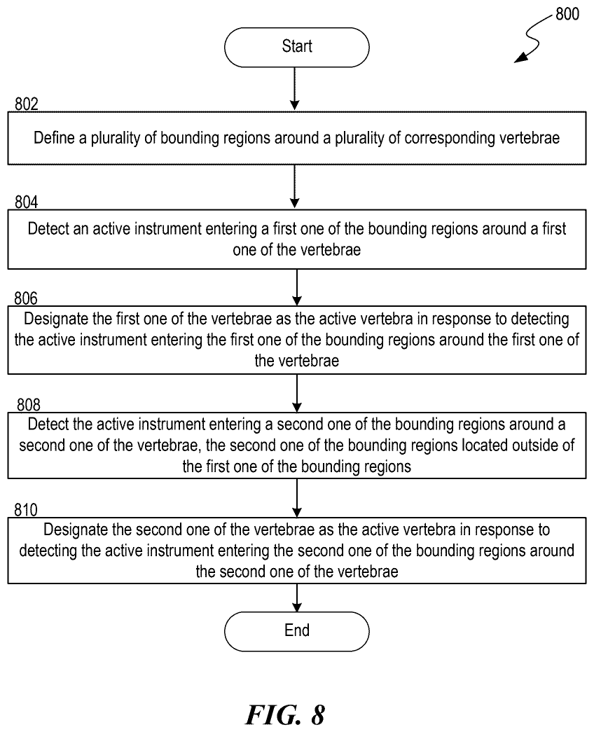

The system 100 dynamically adjusts the active vertebra based on the movement of instruments (such as instrument 708) between the bounding regions 706. Methods of determining the active vertebra are discussed in detail below with reference to FIG. 8. In some embodiments, the bounding regions 706 can be constructed as a mesh with some tolerance, such as 0.1 mm, 1 mm, and/or the like around each of the vertebrae 704b to accommodate slight variations in instrument positioning while ensuring consistency and preventing rapid changes in the active vertebra.

In some embodiments, the system 100 can be adapted to track orthopedic bones throughout surgical procedures or other anatomical structures not limited to bones (e.g., soft tissues, organs, vascular structures). The flexibility allows for broad applications in various surgical fields, improving the accuracy and efficiency of medical interventions.

FIG. 8 is a flow diagram of a process or method 800 for dynamically selecting and managing an active vertebra after detecting an active instrument in the tracking environment 700 of FIG. 7 in accordance with embodiments of the present technology. Although some features of the method 800 are described in the context of the system 100 shown in FIGS. 1-7 for the sake of illustration, one skilled in the art will readily understand that the method 800 can be carried out using other suitable systems 100 and/or devices described herein. Similarly, while reference is made herein to preoperative image data, intraoperative image data, and a surgical scene, the method 800 can be used with other types of information about other scenes. Likewise, implementations and embodiments can include different and/or additional acts or can perform the acts in different orders.

At block 802, the method 800 can include defining a plurality of bounding regions (e.g., the bounding regions 706 in FIG. 7) around a plurality of corresponding vertebrae (e.g., the vertebra 704 in FIG. 7). The process of defining the bounding regions 706 is the same as or similar to defining the bounding region 408 with reference to FIGS. 4A-B. For example, the bounding regions 706 can be defined as a spherical volume around the vertebra, or the bounding regions can be more complex meshes that precisely follow the surface contours of the vertebrae. In some embodiments, proximity thresholds can be defined along with the bounding regions to provide a tolerance to the bounding regions, as discussed further with reference to proximity thresholds in FIG. 5 and FIG. 7. Once the bounding regions are defined, the system 100 can use various tracking technologies to monitor the movement of surgical instruments within these regions, as discussed in further detail above with reference to FIG. 5.

In some embodiments, the bounding regions defined by the method 800 for determining the active vertebra are smaller than those defined by the method 500 for determining the active instrument. The smaller bounding regions around the vertebrae for determining the active vertebra ensure greater accuracy in determining which vertebra is currently active. By defining the bounding regions more narrowly for selection of an active vertebra, the system 100 can more accurately detect when a surgical instrument is in close proximity to a specific vertebra, lowering the risk of mistakenly classifying an adjacent vertebra as the active vertebrae. The level of precision can be important during procedures that require fine manipulations and targeted interventions, such as placing screws or performing localized bone removals. In contrast, the larger bounding regions defined for determining the active instrument provide a broader range of detection. The larger bounding regions can allow the system 100 to recognize when an instrument is within the general vicinity of the vertebra to facilitate smoother transitions between different states of instrument activity. The broader detection can be beneficial for maintaining continuous tracking of the instrument as the instrument moves through the surgical field containing the broader bounding region and approaches the active vertebra, even if the instrument has not yet entered the narrower bounding region of the vertebra.

At block 804, the method 800 can include detecting that an active instrument has entered a first one of the bounding regions around a first one of the vertebrae (e.g., the first vertebra 704 in FIG. 7). When the system 100 identifies that an active instrument has entered a bounding region, the system 100 triggers a series of actions to update the user interface (e.g., user interface 900) and/or provide real-time feedback to the surgeon. Methods of determining the active instrument and detecting the active instrument are discussed with reference to FIGS. 4A-6C.

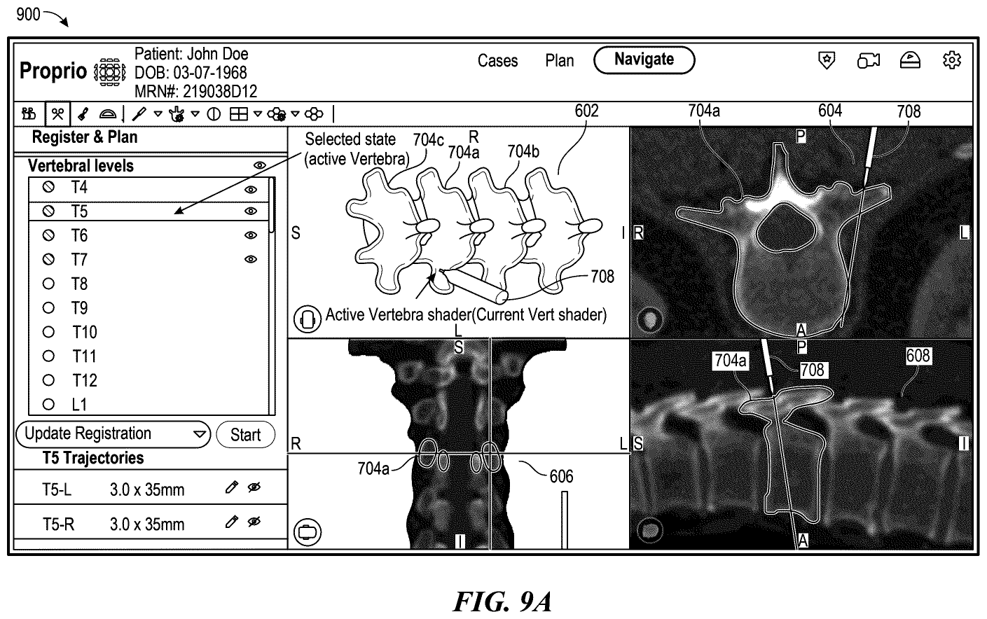

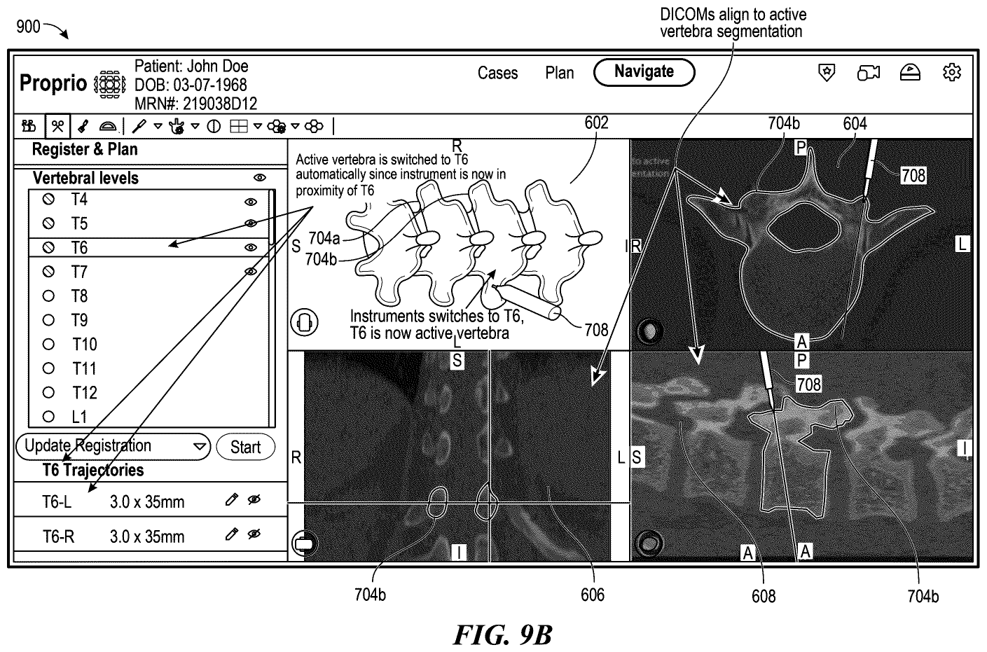

At block 806, the method 800 can include designating the first one of the vertebrae (e.g., first vertebra 704a in FIG. 7) as the active vertebra in response to detecting that the active instrument has entered the first one of the bounding regions around the first one of the vertebrae. Once the active instrument is detected within the first bounding region, the system 100 designates the corresponding vertebra (e.g., the first vertebra 704a) as the current active vertebra of the procedure. For example, FIGS. 9A and 9B illustrate a user interface 900 (e.g., a display) visible to a user of the system via the display device 104 (e.g., a head-mounted display device) and/or the secondary display 226 in accordance with embodiments of the present technology. That is, FIGS. 9A and 9B illustrate operation of the user interface 900 according to the method 800 of FIG. 8. The user interface 900 can include features generally similar or identical to the user interface described in detail above with reference to FIGS. 6A-6C, such as the primary viewport 602 or panel for displaying a 3D view of the one or more of the vertebrae 704 (e.g., an active one of the vertebrae 704 and/or one or more inactive ones of the vertebrae 704) and the secondary panels or viewports 604, 606, 608 each displaying a corresponding different 2D view (e.g., a coronal, sagittal, and/or axial 2D view) one or more of the vertebrae 704.

Referring to FIG. 9A, in the illustrated embodiment the active instrument 708 has entered the bounding region around the first vertebra 704a (e.g., the first bounding region 706a shown in FIG. 7) and the system 100 has therefore designated the first vertebra 704a as the active vertebra. In response to the designation, the system 100 can manipulate the user interface 900 to present data associated with the active vertebra by, for example, highlighting the active first vertebra 704a in one or more of the viewports 602, 604, 606, 608, updating the displayed images to display the active vertebra, and/or adjusting the views or angles of 3D imaging data to provide a clearer and more detailed perspective of the active vertebra. In some embodiments, the user interface 900 can utilize visual cues to emphasize the active vertebra. The visual cues can include changes in color, such as highlighting the active first vertebra 704a in a distinct hue or intensity, and/or employing boundary markers that outline the boundaries of the active first vertebra 704a more prominently. The visual cues can provide feedback to the viewer of the imaging system 100 by identifying the active anatomical structure during the procedure. The real-time adjustment allows the surgeon to maintain situational awareness and make more informed decisions during the procedure. In some embodiments, the system 100 can provide haptic feedback and/or audible alerts to notify the surgeon that the instrument has entered a new bounding region. Further, the system 100 can log the entry of the active instrument into the bounding region of the active vertebra for later review or recordkeeping. For example, FIG. 9A displays an overlaid representation of an active vertebra (e.g., the first vertebra 704a in FIG. 9A), and illustrates the display of the 3D and 2D image data as the instrument 708, approaches, enters, moves through, and/or exits a bounding region (e.g., the first bounding region 706a of FIG. 7) around the first vertebra 704 in accordance with embodiments of the present technology. The user interface 900 can operate by continuously updating the displayed 3D image data based on the real-time movements of the instrument 708 within the tracking region and bounding regions defined around each vertebra.

At block 808, the method 800 can include detecting that the active instrument has entered a second one of the bounding regions around a second one of the vertebrae, the second one of the bounding regions located outside of the first one of the bounding regions. When the system 100 detects the active instrument transitioning from the first bounding region to the second, the system 100 can infer that the focus of the procedure is shifting from one vertebra to another. The detection of the active instrument entering the second bounding region can be completed using the same or similar methods as that of detecting the active instrument entering the first bounding region.

At block 810, the method 800 can include designating the second one of the vertebrae as the active vertebra in response to detecting that the active instrument has entered the second one of the bounding regions around the second one of the vertebrae. Upon detecting that the active instrument in the second bounding region, the system 100 reassigns the active vertebra from the first one of the vertebrae to the second one of the vertebrae. Reassigning the active vertebra status can cause the user interface to shift to highlight the second vertebra, update the displayed 3D and/or images and/or adjust other relevant data to focus on the second one of the vertebrae (e.g., the new active vertebra).

For example, referring to FIG. 9B, the instrument 708 has subsequently entered the bounding region of the second vertebra 704b (e.g., the second bounding region 706b shown in FIG. 7). The system 100 can detect the movement (e.g., block 808 in FIG. 8) and automatically update the active vertebra, switching from the first vertebra 704a to the second vertebra 704b. The user interface 900 can reflect the change by highlighting the second vertebra 704b as the new active vertebra or otherwise indicating the change (e.g., via visual, auditory, and/or haptic feedback). In some embodiments, the user interface 900 can include additional features such as zoom, rotation, and/or annotations. The ability to switch the active vertebra based on the proximity of the instrument ensures that the system 100 remains responsive to the surgeon's movements. The responsiveness is particularly important in complex surgeries involving multiple vertebrae, where precise navigation and real-time updates are essential for successful outcomes. By automatically updating the active vertebra, the system 100 decreases the need for manual adjustments and allows the surgeon to focus on the task at hand.

In some embodiments, the system 100 can determine the spatial relationship between the active instrument and anatomical reference planes of the active vertebra. For example, the system 100 can detect which side of a vertebra the active instrument is positioned relative to anatomical planes such as the sagittal plane, coronal plane, and/or axial plane. The system 100 (e.g., via the processing device 102) can use the previously-captured image data (e.g., CT scan data, MRI scan data) to define anatomical reference planes for each of the vertebrae 704. More specifically, the registration processing device 105 can register the previously-captured image data to the real-time images captured by the cameras 112 to establish a coordinate system that includes anatomical reference planes aligned with the vertebrae 704. The tracking processing device 107 can determine the position of the active instrument (e.g., instrument 708) relative to these anatomical reference planes by comparing the 3D coordinates of the active instrument to the plane equations defining the reference planes. For example, the system 100 can determine whether the active instrument is positioned on the left side or right side of the sagittal plane of the active vertebra, anterior or posterior to the coronal plane, and/or superior or inferior to the axial plane.

This spatial relationship information determined by the system 100 can be displayed on the user interface 900 to provide additional context to the viewer of the imaging system 100. In some embodiments, the user interface 900 can include visual indicators (e.g., color coding, directional arrows, text labels) that identify the spatial relationship between the active instrument and the anatomical reference planes of the active vertebra. In some embodiments, the system 100 can automatically adjust the displayed 2D views in the secondary viewports 604, 606, 608 based on the spatial relationship between the active instrument and the anatomical reference planes to provide the corresponding cross-sectional views for the current instrument position.

In some embodiments, the system 100 provides near-real-time or real-time feedback to alert the surgeon or other user if the active instrument crosses predetermined anatomical boundaries or approaches particular anatomical structures. In some embodiments, the system 100 records and stores the spatial relationship data between the active instrument and the anatomical reference planes throughout at least a portion of the surgical procedure (e.g., to be used in post-operative analysis). The stored spatial relationship data can include timestamps, instrument positions relative to anatomical planes, movement trajectories, duration of instrument presence in specific anatomical regions, and/or the like. For example, the stored spatial relationship data can be used to generate post-operative reports that indicate a time period spent on particular areas of anatomical structures. In some embodiments, the post-operative reports can be used to compare surgical approaches across different procedures, identify best practices, and/or provide feedback to surgeons for users of the system 100.

### III. Additional Examples

The following examples are illustrative of several embodiments of the present technology:

1. A method of selecting three-dimensional (3D) image data configured to be displayed on a user interface, the method comprising:
defining a bounding region around a vertebra;
detecting that a first instrument has entered into the bounding region;
in response to detecting that the first instrument has entered into the bounding region, designating the first instrument as an active instrument;
detecting that a second instrument has entered into the bounding region while the first instrument is within the bounding region;
maintaining the first instrument as the active instrument in response to detecting that the second instrument has entered into the bounding region while the first instrument is within the bounding region;
detecting that the second instrument has entered into the bounding region and that the first instrument has moved outside of the bounding region;
designating the second instrument as the active instrument in response to detecting that the second instrument is within the bounding region and the first instrument is outside of the bounding region; and
displaying the 3D image data including a representation of the active instrument on the user interface.

2. The method of example 1, wherein the method further comprises displaying a cross-section of the 3D image data based on a position and/or orientation of the active instrument.

3. The method of example 1 or example 2, wherein the bounding region comprises a proximity threshold, the proximity threshold configured to determine that the active instrument is within the bounding region based on the active instrument located within a predefined distance from the bounding region.

4. The method of any one of examples 1-3, wherein displaying the 3D image data includes highlighting at least a portion of the representation of the active instrument on the user interface.

5. The method of any one of examples 1-4, wherein the first instrument and/or the second instrument comprise a surgical tool, a surgical implant, or a surgical tool coupled to a surgical instrument.

6. The method of any one of examples 1-5, wherein the bounding region is based on a 3D mesh of the vertebra.

7. The method of example 6, wherein the method further comprises updating the bounding region to reflect a change in dimension of the vertebra.

8. The method of any one of examples 1-7, wherein the bounding region around the vertebra is defined based on a predefined shape around the vertebra.

9. A method of selecting three-dimensional (3D) image data configured to be displayed on a user interface, the method comprising:
defining a plurality of bounding regions around a plurality of corresponding vertebrae;
detecting that an active instrument has entered a first one of the bounding regions around a first one of the vertebrae;
designating the first one of the vertebrae as the active vertebra in response to detecting that the active instrument has entered the first one of the bounding regions around the first one of the vertebra;
detecting that the active instrument has entered a second one of the bounding regions around a second one of the vertebrae, the second one of the bounding regions located outside of the first one of the bounding regions;
designating the second one of the vertebrae as the active vertebra in response to detecting that the active instrument has entered the second one of the bounding regions around the second one of the vertebrae; and
displaying the 3D image data including a representation of the active vertebrae on the user interface.

10. The method of example 9, wherein the method further comprises displaying a cross-section of the 3D image data.

11. The method of example 9 or example 10, wherein the bounding region comprises a proximity threshold, the proximity threshold configured to determine that the active instrument is within the bounding region based on the active instrument located within a predefined distance from the bounding region.

12. The method of any one of examples 9-11, wherein displaying the 3D image data includes highlighting at least a portion of the representation of the active vertebra on the user interface.

13. The method of any one of examples 9-12, wherein the instrument is a surgical tool, a surgical implant, or a surgical tool coupled to a surgical instrument.

14. The method of any one of examples 9-13, wherein the plurality of bounding regions around the plurality of corresponding vertebrae are each defined as a predefined shape around each of the plurality of corresponding vertebrae.

### IV. CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

We claim:

1. A method of selecting three-dimensional (3D) image data configured to be displayed on a user interface, the method comprising:
defining a plurality of bounding regions around a plurality of corresponding vertebrae;
detecting that a first instrument has entered a first one of the bounding regions around a first one of the vertebrae;
in response to detecting that the first instrument has entered into the first one of the bounding regions around the first one of the vertebra:
designating the first instrument as an active instrument, and
designating the first one of the vertebrae as an active vertebra;
detecting that the first instrument has entered a second one of the bounding regions around a second one of the vertebrae, the second one of the bounding regions located outside of the first one of the bounding regions;
in response to detecting that the first instrument has entered the second one of the bounding regions around the second one of the vertebrae:
maintaining the first instrument as the active instrument, and
designating the second one of the vertebrae as the active vertebra;
detecting that the second instrument has entered into the second bounding region and that the first instrument has moved outside of the second bounding region;
in response to detecting that the second instrument is within the second bounding region and the first instrument is outside of the second bounding region, designating the second instrument as the active instrument; and
displaying the 3D image data including a representation of the active instrument and the active vertebrae on the user interface.

2. The method of claim 1, further comprising displaying a cross-section of the 3D image data based on a position and/or orientation of the active instrument.

3. The method of claim 1, wherein the bounding regions comprise proximity thresholds, the proximity thresholds configured to determine that the active instrument is within a bounding region based on the active instrument being located within a predefined distance from the bounding region.

4. The method of claim 1, wherein displaying the 3D image data includes highlighting at least a portion of the representation of the active instrument and/or the active vertebra on the user interface.

5. The method of claim 1, wherein the first instrument and/or the second instrument comprise a surgical tool, a surgical implant, or a surgical tool coupled to a surgical instrument.

6. The method of claim 1, wherein each bounding region around a vertebra is defined based on a predefined shape around the vertebra.

7. A method of selecting three-dimensional (3D) image data configured to be displayed on a user interface, the method comprising:
defining a bounding region around a vertebra;
detecting that a first instrument has entered into the bounding region;
in response to detecting that the first instrument has entered into the bounding region, designating the first instrument as an active instrument;
detecting that a second instrument has entered into the bounding region while the first instrument is within the bounding region;
maintaining the first instrument as the active instrument in response to detecting that the second instrument has entered into the bounding region while the first instrument is within the bounding region;
detecting that the second instrument has entered into the bounding region and that the first instrument has moved outside of the bounding region;
designating the second instrument as the active instrument in response to detecting that the second instrument is within the bounding region and the first instrument is outside of the bounding region; and
displaying the 3D image data including a representation of the active instrument on the user interface.

8. The method of claim 7, wherein the method further comprises displaying a cross-section of the 3D image data based on a position and/or orientation of the active instrument.

9. The method of claim 7, wherein the bounding region comprises a proximity threshold, the proximity threshold configured to determine that the active instrument is within the bounding region based on the active instrument located within a predefined distance from the bounding region.

10. The method of claim 7, wherein displaying the 3D image data includes highlighting at least a portion of the representation of the active instrument on the user interface.

11. The method of claim 7, wherein the first instrument and/or the second instrument comprise a surgical tool, a surgical implant, or a surgical tool coupled to a surgical instrument.

12. The method of claim 7, wherein the bounding region is based on a 3D mesh of the vertebra.

13. The method of claim 12, wherein the method further comprises updating the bounding region to reflect a change in dimension of the vertebra.

14. The method of claim 7, wherein the bounding region around the vertebra is defined based on a predefined shape around the vertebra.

15. A method of selecting three-dimensional (3D) image data configured to be displayed on a user interface, the method comprising:
defining a plurality of bounding regions around a plurality of corresponding vertebrae;
detecting that an active instrument has entered a first one of the bounding regions around a first one of the vertebrae;
designating the first one of the vertebrae as the active vertebra in response to detecting that the active instrument has entered the first one of the bounding regions around the first one of the vertebra;
detecting that the active instrument has entered a second one of the bounding regions around a second one of the vertebrae, the second one of the bounding regions located outside of the first one of the bounding regions;
designating the second one of the vertebrae as the active vertebra in response to detecting that the active instrument has entered the second one of the bounding regions around the second one of the vertebrae; and
displaying the 3D image data including a representation of the active vertebrae on the user interface.

16. The method of claim 15, wherein the method further comprises displaying a cross-section of the 3D image data.

17. The method of claim 15, wherein the bounding region comprises a proximity threshold, the proximity threshold configured to determine that the active instrument is within the bounding region based on the active instrument located within a predefined distance from the bounding region.

18. The method of claim 15, wherein displaying the 3D image data includes highlighting at least a portion of the representation of the active vertebra on the user interface.

19. The method of claim 15, wherein the instrument is a surgical tool, a surgical implant, or a surgical tool coupled to a surgical instrument.

20. The method of claim 15, wherein the plurality of bounding regions around the plurality of corresponding vertebrae are each defined as a predefined shape around each of the plurality of corresponding vertebrae.
